(19) United States
(12) Patent Application Publication
(10) Pub. No.: US 2007/0223651 A1
(43) Pub. Date: Sep. 27, 2007

(54) Dual modality mammography device

(76) Inventors: Douglas Jay Wagenaar, South Barrington, IL (US); A Hans Vija, Evanston, IL (US); Ansgar Graw, Chicago, IL (US); Eric G. Hawman, Schaumburg, IL (US); Jinhun Joung, Algunquin, IL (US)

Correspondence Address: SIEMENS CORPORATION;INTELLECTUAL PROPERTY DEPARTMENT, 170 WOOD AVENUE SOUTH, ISELIN, NJ, 08830, US

(21) Appl. No.: 11/385,325

(22) Filed: Mar. 21, 2006

(51) Int. Cl. A61B 6/04

(52) U.S. Cl. 378/37

(57) ABSTRACT

A medical imaging system locates tumors in tissue surrounding an anatomical portion of the human body subject to investigation for primary lesions. The imaging system comprises a single-photon limited angle tomographic imaging device to provide a first emission image including tissue surrounding the anatomical portion. In combination with the single photon limited angle tomographic imaging device, a digital x-ray device provides an x-ray transmission image of the anatomical portion and tissue thereabout. A third component of the imaging system is a single-photon nuclear imaging device for tomographic image acquisition and reconstruction to produce a second emission image of the anatomical portion and tissue previously viewed by the digital radiography device. Registration of the first emission image, the transmission image and the second emission leads to selection of two- and three-dimensional image sets of the anatomical portion and tissue surrounding the anatomical portion.

### TECHNICAL FIELD

The present invention relates generally to the field of multi-modality imaging systems and devices such as those used in medical diagnoses. More particularly the invention relates to registration of medical images obtained from the dual modalities of x-ray computer assisted tomography (CT) and single photon emission computed tomography (SPECT) using the latter in conventional and limited angle imaging modes to provide simultaneous computerized graphical display of all relevant anatomical and physiological information associated with a primary lesion and surrounding tissue.

### BACKGROUND ART

The use of multi-modality diagnostic imaging systems combining x-ray transmission data with radionuclide emission data provides visual information of both anatomical structure and physiological function of patients subject to diagnosis of disease. X-ray transmission (CT) imaging provides anatomical images that are complemented by radionuclide imaging following injection of a radio-labeled material into the patient's bloodstream. The radio-labeled material concentrates in an organ or lesion or interest. At a prescribed time after injection, a pattern of gamma rays corresponding to the concentration of the radio-labeled material may be imaged using a rectilinear scanner, scintillation camera, SPECT or a positron emission tomography (PET) system. Detectors used in these imaging systems respond to gamma radiation from the patient, collecting data used to form three-dimensional images e.g. SPECT images or tomographic images of the distribution of radioisotope within the patient.

A radio-nuclide imaging procedure requires a means to define the path along which an emitted gamma ray travels towards an imaging detector. In SPECT systems, for example, a collimator (typically made of lead) placed between the patient and the detector establishes the path, from the site where radio-labeled material concentrates to the detector, along which a gamma ray will travel.

Anatomical modeling of the human body with a computer depends on a method for the accurate registration and congruence of multi-dimensional, multi-modality image sets synthesized into a single composite multi-valued image. The image forming process involves mapping of an object to visualize the object and its properties in terms of structure and function. Multi-modality images provide enhanced capability for visualization and quantitative analysis of biomedical structures. Spatial registration of medical images, obtained from several modalities, such as PET, SPECT and CT and the like, allow direct visualization for study of the structure and function of internal organs.

Previous studies in this area include the dual mode stereotactic localization method described in U.S. Pat. No. 6,389,098 using the structural digital X-ray image provided by conventional stereotactic core biopsy instruments with the additional functional metabolic gamma imaging obtained with a dedicated compact gamma imaging mini-camera. Before the procedure, the patient is injected with an appropriate radiopharmaceutical. The radiopharmaceutical uptake, expressed by the intensity of gamma emissions, compared (co-registered) with the digital mammography (X-ray) image yields a much smaller number of false positives than would be produced using X-ray images alone. Similar use of nuclear medicine (scinti-mammography) and X-ray techniques, as described in U.S. Pat. No. 6,424,693 provides breast lesion localization results of greater accuracy than earlier methods having the limitation of a single imaging technique.

Although there is a reduction of false positives and improvement in the accuracy of lesion localization according to previous use of multi modality imaging, there remains a need for data to show not only images of primary lesions but also to provide visual information of areas of tissue surrounding the primary lesion.

### SUMMARY OF THE INVENTION

The present invention in its several disclosed embodiments alleviates the drawbacks described above with respect to conventional mammograms and incorporates several additionally beneficial features.

The current invention in its several disclosed embodiments provides a multi-modality imaging device including a limited angle single photon emission computed tomography (SPECT) device that adds data to that obtained by a combination of x-ray digital imaging and SPECT imaging. Collection of multi-modality imaging data provides a diagnosing radiologist with a reconstruction of registered images showing detail of an imaged portion of a patient's body, that may include a primary lesion, and tissue surrounding the imaged portion. Knowledge of the relative geometry of the limited angle SPECT device with the combined digital imaging and SPECT devices allows computerized graphical display of all relevant tissues simultaneously.

A multi-modality technique, including digital mammography (X-ray) and limited angle SPECT according to the present invention, used to investigate breast cancer, for example, gives the radiologist an image of both sides of the thorax providing evidence of the condition of breast tissue and surrounding tissue of the axillary and mediastinal regions that may be affected by metastatic breast carcinoma. The invention provides the radiologist with a complete view of the anatomical location of emission imaging (SPECT) “hot spots” associated with abnormalities, neoplasms and stage 2 metastases suggesting the possibility of cancer in regions surrounding the breast.

Through the use of image registration techniques, the radiologist also obtains a correlation between conventional digital mammographic views and views covering the breast and surrounding tissue derived from the two modes of SPECT scanning. Computer enhancement of abnormalities, seen in X-ray images, using tomosynthesis and SPECT data, produces high resolution images suitable for viewing at a computer workstation to compare the abnormalities with potentially cancerous sites revealed from SPECT images. Multi modality tomography combining X-ray CT and SPECT modalities provides benefits to the study of breast cancer and possibly prostate cancer in a manner similar to PET added to CT in general oncology.

More particularly the present invention provides a medical imaging system for locating tumors in tissue surrounding an anatomical portion of the human body subject to investigation for primary lesions. The imaging system comprises a single-photon limited angle tomographic imaging device for tomographic image acquisition and reconstruction to provide a first emission image including tissue surrounding the anatomical portion. In combination with the single photon limited angle tomographic imaging device, a digital radiography device includes an X-ray generator, X-ray tube, and a digital X-ray detector to produce an X-ray transmission image of the anatomical portion and tissue thereabout. A third component of the imaging system is a single- photon nuclear imaging device for tomographic image acquisition and reconstruction to produce a second emission image of the anatomical portion and tissue previously viewed by the digital radiography device, wherein the first emission image, the transmission image and the second emission image combine to create two- and three-dimensional, co-registered image sets for a computer graphical display of the anatomical portion and tissue surrounding the anatomical portion.

The beneficial effects described above apply generally to the exemplary devices and mechanisms disclosed herein of the dual modality mammography device. The specific structures through which these benefits are delivered will be described in detail hereinbelow.

### BRIEF DESCRIPTION OF THE DRAWINGS

The invention will now be described in greater detail in the following way of example only and with reference to the attached drawings, in which:

FIG. 1 is a perspective view taken from the side of a multi-modality imaging system according to the present invention.

FIG. 2 is a perspective view similar to that of FIG. 1 showing an arrangement of an x-ray digital detector deployed in a plane substantially at right angles to the plane of a radionuclide detector such as a gamma camera.

FIG. 3 is a perspective view of an imaging system according to the present invention showing a second nuclear detector used for data acquisition of tissue surrounding a primary lesion.

FIG. 4 is a schematic representation of a collimator used with single photon emission computed tomography to provide limited angle tomographic data.

### DETAILED DESCRIPTION OF THE INVENTION

As required, detailed embodiments of the present invention are disclosed herein; however, it is to be understood that the disclosed embodiments are merely exemplary of the invention that may be embodied in various and alternative forms. The figures are not necessarily to scale; some features may be exaggerated or minimized to show details of particular components. Therefore, specific structural and functional details disclosed herein are not to be interpreted as limiting, but merely as a basis for the claims and as a representative basis for teaching one skilled in the art to variously employ the present invention.

The present invention combines X-ray and nuclear medicine multi-modality imaging techniques, particularly emphasizing SPECT imaging, used in conventional and limited angle scanning modes, to assess a target portion (imaged object) of a person's body for the presence of primary lesions and lymph node metastases associated with cancerous tumors. While the present invention exemplifies its use in the field of mammography, it will be appreciated that equipment and processes described herein have application in other areas of diagnostic medical investigation. Imaging equipment is multi-modality and takes advantage of existing and developing image display routines associated with combined scanning techniques including Positron Emission Tomography (PET)/Computed Tomography (CT), SPECT/CT, and PET/Magnetic Resonance (MR).

Acquisition of mammogram datasets according to the present invention occurs after placing a patient in the conventional mammographic position for recording digital X-ray mammography scans and nuclear scans using a planar detector. However, image enhancement of conventional mammographic images occurs through the use of digital tomosynthesis, which introduces a depth component that makes breast cancers easier to see in dense breast tissue. This depth information used with resolution recovery techniques, such as Siemens TM Flash 3D, provides enhanced images obtained by nuclear scanning. Further breast image improvement is possible by application of attenuation and scatter correction to reconstructed images.

Tomosynthesis differs from standard mammography in the same way as a CT scan differs from a standard X-ray procedure. In tomosynthesis, during a seven second examination the X-ray tube moves in a 50-degree arc as it scans eleven low-dose images around the patient's breast. A computer then assembles the image data to provide high-resolution cross-section and three-dimensional images viewable by the radiologist at a computer workstation.

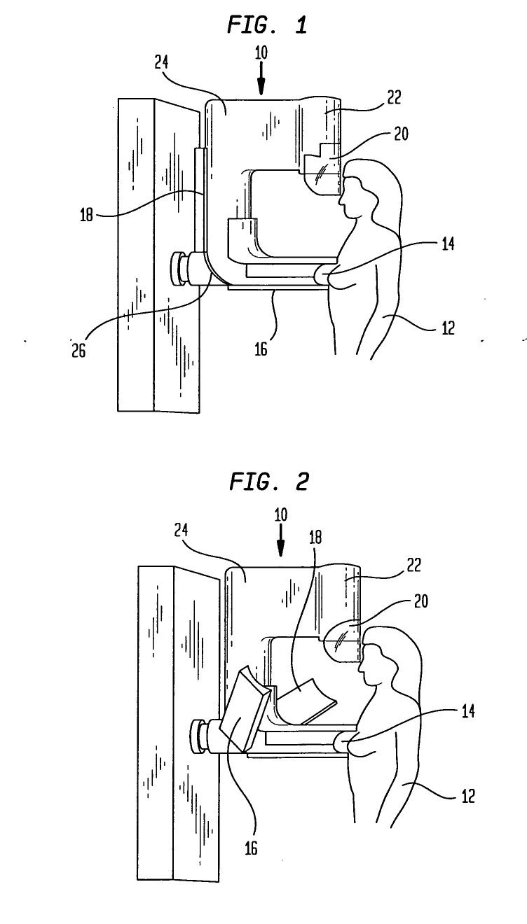

Referring to the figures, wherein like numbers refer to like parts throughout the several views, FIG. 1 shows a multi-modality imaging system 10 having an appearance similar to that of a conventional X-ray mammography unit. An imaging system 10 according to the present invention reveals the presence of tumors or other defects in breast tissue and surrounding tissue of a patient who exhibits symptoms associated with cancerous lesions. The imaging system 10 first requires that the patient 12 (surrogate image) adopt a position for collection of data from the area of breast 14 under study, also referred to herein as the imaged object 14. Data collection proceeds using a dual modality technique involving sequential acquisition of transmission signals and emission signals for sensing by detectors 16, 18 that provide data to a computer (not shown). The computer processes the data into images for display on a monitor or a flat screen liquid crystal display (LCD).

Dual modality mammography according to the present invention uses geometry like that of conventional digital or film-based mammography. The lower detector 16 is a digital mammography plate. Near the patient's head, an additional radiation shield 20 protects the patient's face from the X-ray tube 22. Acquisition of digital tomosynthesis data occurs during scanning of the imaged object 14, when X-rays pass through the imaged object 14 at known radiation intensity. Movement of the X-ray tube 22 and the digital mammographic plate detector 16 relative to each other accompanies measurement of the intensity of radiation detected at different projection angles to produce tomosynthesis data as the gantry 24 of the imaging system 10 follows the scanning arc over the imaged object 14. Digital tomosynthesis data allows the use of depth information for enhancing and correcting nuclear medicine data.

In one embodiment according to the present invention, detectors 16, 18 suitable for image data collection include an X-ray computed tomography (CT) device operating in transmission mode to collect anatomical data. Using digital X-ray CT, a beam of X-rays impinges on a flat panel detector 16 that uses an active matrix of amorphous silicon pixels to detect transmitted X-rays. The flat panel detector converts X-ray signals into electrical signals for image generation after amplifying and digitizing the electrical signals.

An imaging system 10 according to the present invention also uses a nuclear medicine (NM) imaging device such as single photon emission computed tomography (SPECT) or positron emission tomography (PET) for collecting functional data of the imaged object 14. A planar detector 18, suitable for nuclear medicine imaging, is a gamma camera that accumulates counts of gamma photons absorbed by a crystal in the camera. The crystal scintillates, emitting a faint flash of light in response to incident gamma radiation. Photomultiplier tubes (PMT) behind the crystal detect the fluorescent flashes and the computer sums the fluorescent counts. Alternatively, the gamma camera may be based on a solid-state radiation detector, such as CZT (Cadmium Zinc Telluride). A solid-state camera may be made more compact than one based on conventional scintillator and photomultiplier tube technology. The computer in turn constructs and displays a two dimensional image of the relative spatial count density on the monitor or LCD. This image then reflects the distribution and relative concentration of radioactive tracer elements present in the imaged object 14, i.e. the patient's breast.

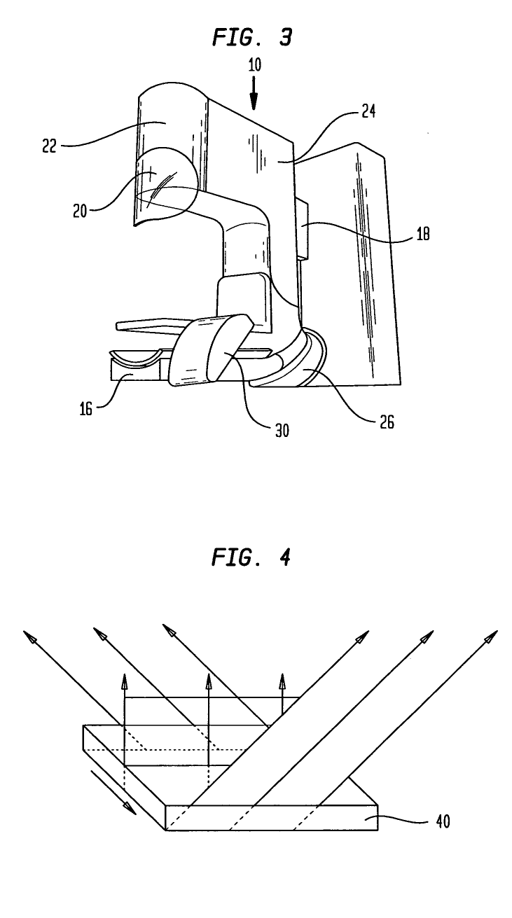

FIG. 2, shows the dual modality mammography imaging system 10 in a condition intermediate between the recording of a digital X-ray scan using the flat panel detector 16 and collection of emission data using the planar detector 18 to sense gamma photons. The X-ray detector 16 and the gamma photon detector 18 have a design placing them at right angles to each other in an L-shaped structure including a mounting 26 for pivotal movement that places the flat plate detector 16 in position to collect scan data while the planar detector 18 remains stored behind the gantry 24, as shown in FIG. 1 and FIG. 3. Rotation of the detectors 16, 18 using the pivoting mounting 26 exchanges the digital X-ray mammographic plate or flat panel detector 16 with the nuclear medicine planar detector 18. Other equipment settings and patient positioning remain unchanged during exchange of the detectors 16, 18. Carefully engineered tolerances allow substantially precise alignment between two data sets obtained during X-ray and NM scans.

FIG. 3: is a perspective view showing an imaging system 10 according to the present invention providing illustration of a second nuclear detector 30 used to collect tomosynthesis data from the axilla and mediastinal regions of the patient's body. The second nuclear detector 30 occupies a position adjacent to the location of either the digital mammography plate 16 or the gamma photon planar detector 18, depending on which of these detectors 16, 18 is in use. Scanning devices within the second nuclear detector 30 collect image data for either the axilla (underarm) or the mediastinum (sternum) region of the patient, according to the positioning of the patient relative to the second nuclear detector 30, the surface of which makes contact with the woman's skin. Behind this surface, collimator and/or detector elements are moved for the purpose of acquiring tomographic projection data. The surface 30 may be cylindrical, spherical, or of a more general convex shape to minimize the distance between the tissue to be examined and the detector. A conformal shape also will be more comfortable for the patient. From this position, manipulation of data from the second nuclear detector 30 provides images that may have a body outline superimposed upon them.

As indicated previously, the second nuclear detector 30 includes at least one rotating or scanning device 40 (see e.g. FIG. 4) to acquire limited angle tomographic datasets for image reconstruction. The manifestation of breast cancer in the axilla and the mediastinal regions is typically a small, potentially cancerous site (“hot spot”) in the form of a radiopharmaceutical absorbing, metastatic lymph node. Limited angle tomographic sampling techniques usually suffice to detect and render these sites in the image space.

FIG. 4 provides a schematic representation of the scanning device 40, preferably a specially-designed collimator that presents the second nuclear detector 30 with a number of tomographic views of tissue in the axillary and mediastinal tissues surrounding the imaged object 14. It is known that a collimator comprises a radiation absorbing material (typically lead or similar high density material) that provides passageways through which gamma rays pass from a site of radionuclide absorption to a nuclear detector 30. The present invention provides a collimator wherein the passageways change continuously from one angle on one end to a different angle at the other end of the collimator. Variation of angles, from one end of the collimator to the other, presents the second nuclear detector 30 with substantially different tomographic views of tissue in contact with the detector 30 as the tissue moves with respect to the collimator. An equivalent effect occurs when the imaged tissue remains in one position as the collimator scans the tissue. This collimator is described in copending patent application Ser. No. (2005P01554US), assigned to the same assignee herein.

A varying slant angle collimator according to the present invention produces image data from different scans used to reconstruct limited angle tomographs of axillary and mediastinal tissue surrounding the imaged object 14. In combination with the X-ray scan and emission scan data, which primarily collect information of the imaged object 14 as described previously, data obtained by the scanning device 40 augments available data so that image reconstructions include the imaged object 14 and surrounding tissue containing axillary and mediastinal regions. A further advantage of the imaging system 10 and related processes is the fact that X-ray digital mammography data, combined with data from conventional and limited angle SPECT scans, gives diagnostic information acquired by scanning the imaged object 14 of a patient's breast and surrounding tissue in axillary and mediastinal regions. All three modes of scanning, i.e. X-ray transmission CT, conventional SPECT and limited angle SPECT, proceed within the same imaging session, following a single injection of radioactive tracer.

As an alternative to the varying slant angle collimator, a bilateral collimator, such as disclosed in U.S. Pat. No. 4,659,935, issued to Eric G. Hawman, assigned to the same assignee herein, and incorporated herein in its entirety by reference, also may be used to obtain tomographic data for SPECT mammography in accordance with the present invention.

An embodiment of the present invention is an imaging system 10 comprising three independent imaging devices operating together to create two- and three-dimensional, co-registered image sets of the imaged object 14 of the human female breast and tissue surrounding the imaged object 14.

A distinguishing feature of the present invention is the use of a single-photon (SPECT) limited angle tomographic imaging device 30 that is designed to provide complementary, 3-D imaging of axillary and mediastinal regions of tissue surrounding the breast (i.e. the imaged object 14).The limited angle tomographic imaging device 30 collects data to augment that obtained using X-ray digital mammography based on equipment comprising a generator of X-rays, an X-ray tube 22, and a digital mammography flat plate detector 16. This flat plate, X-ray detector 16 operates in “tomosynthesis” mode, creating tomographs and 3-dimensional estimates of ductal, cystic, tumor and other physical structures in the region of the breast 14. The third component of the imaging system 10 is a second SPECT device that scans breast tissue previously viewed by the X-ray device. Substitution of the gamma photon planar detector 18 for the flat plate X-ray detector 16 facilitates collection of emission projection data for processing and image reconstruction by the computer of the imaging system 10. The design of the imaging system 10 establishes known relative geometry among the two SPECT devices and the X-ray digital mammography device. Using known image registration techniques, the computer processes data obtained through separate scans of the imaged object 14 to provide a reconstructed 3-D image display of the breast tissue and axillary, mediastinal regions surrounding the breast.

A method according to the present invention addresses the needs of female patients who have known or suspected breast cancer. Initial patient analysis includes a record of genetic and proteomic signatures to indicate the sub-type of breast cancer that might be present. The genetic and proteomic signature information determines the radiopharmaceutical agent administered to the patient by injection. The pharmaceutical agent circulates through the patient's body for sufficient time to allow its absorption by tumors and lesions. When this time expires, the patient undergoes X-ray and nuclear scans from which to create a comprehensive image dataset that combines the axilla, and mediastinal regions and the two breasts with an estimate of the skin surface in contact with the detector 30 surface, plus scatter detected in the nuclear scans. This comprehensive image set allows the radiologist to select image attributes, e.g. 3-D images, and to magnify portions of the displayed image for closer scrutiny.

As described, the imaging system 10 of the present invention comprises X-ray mammography, which is widely accepted as the primary screening and follow-up imaging tool used to study breast cancer. Confidence in the clinical use of this tool opens up opportunities to explore other techniques, such as nuclear medical imaging, to augment and enhance biological and functional aspects of the X-ray mammograms. The present invention is readily adaptable to conventional breast imaging technology because its use requires only minor modification of current mammography equipment.

The imaging system 10 and process described previously also has application for investigating other types of cancer. Image improvements using X-ray tomography with nuclear tomography, as described herein, could improve the quality of prostate imaging. Suitable accommodation for the different region of anatomy, containing the prostate gland, leads to the combined use of X-ray scanning with one nuclear detector for primary target imaging and a second nuclear detector using limited angle tomography to add images of tissue surrounding the primary target.

A dual modality mammography device and its components have been described herein. These and other variations, which will be appreciated by those skilled in the art, are within the intended scope of this invention as claimed below. As previously stated, detailed embodiments of the present invention are disclosed herein; however, it is to be understood that the disclosed embodiments are merely exemplary of the invention that may be embodied in various forms.

What is claimed and desired to be secured by Letters Patent is as follows:

1. A medical imaging system for locating tumors in tissue surrounding an anatomical portion of the human body subject to investigation for primary lesions, wherein the imaging system comprises:
a single-photon limited angle tomographic imaging device for tomographic image acquisition and reconstruction to provide a first emission image including tissue surrounding the anatomical portion;
a digital radiography device including an X-ray generator, X-ray tube, and a digital X-ray detector operable to produce, via tomosynthesis, an X-ray transmission image of the anatomical portion and tissue thereabout; and
a single-photon nuclear imaging device for tomographic image acquisition and reconstruction to produce a second emission image of the anatomical portion and tissue previously viewed by the digital radiography device, wherein the first emission image, the transmission image and the second emission image combine to create two- and three-dimensional, co-registered image sets for a computer graphical display of the anatomical portion and tissue surrounding the anatomical portion.

2. The medical imaging system of claim 1, wherein the anatomical portion is a female breast.

3. The medical imaging system of claim 2, wherein tissue surrounding the female breast includes axillary and mediastinal tissue.

4. The medical imaging system of claim 3, wherein the computer graphical display provides a 3-dimensional simulation of the axillary, mediastinal and breast regions to reveal existence of any primary breast lesions and any lymph node metastases associated with the axillary and the mediastinal tissue.

5. The medical imaging system of claim 1, wherein the single photon limited angle tomographic imaging device includes a collimator having a focus which is located elsewhere than the centerline of the collimator.

6. The medical imaging system of claim 5, wherein the collimator is a fan beam collimator.

7. The medical imaging system of claim 5, wherein the collimator is a bilateral collimator.

8. The medical imaging system of claim 5, wherein the collimator is a varying slant angle collimator.

9. A medical imaging process for locating tumors in tissue surrounding an anatomical portion of the human body subject to investigation for primary lesions, wherein the process comprises:
using a single-photon limited angle tomographic imaging device for tomographic image acquisition and reconstruction to provide a first emission image including tissue surrounding the anatomical portion;
using a digital radiography device including an X-ray generator, X-ray tube, and a digital X-ray detector operable to produce, via tomosynthesis, an X-ray transmission image of the anatomical portion and tissue thereabout;
using a single-photon nuclear imaging device for tomographic image acquisition and reconstruction to produce a second emission image of the anatomical portion and tissue previously viewed by the digital radiography device; and
co-registering the first emission image, the transmission image and the second emission image to create two- and three-dimensional, image sets for a computer graphical display of the anatomical portion and tissue surrounding the anatomical portion.

10. A medical imaging process as claimed in claim 9, wherein the step of using a single-photon nuclear imaging device further comprises the step of using a fan beam collimator.

11. A medical imaging process as claimed in claim 9, wherein the step of using a single-photon nuclear imaging device further comprises the step of using a bilateral collimator.

12. A medical imaging process as claimed in claim 9, wherein the step of using a single-photon nuclear imaging device further comprises the step of using a varying slant angle collimator.
